(19) United States
(12) Patent Application Publication
(10) Pub. No.: US 2003/0171660 A1
(43) Pub. Date: Sep. 11, 2003

(54) System and method for obtaining patient information

(76) Inventor: Thomas V. Foster JR., Eugene, OR (US)

Correspondence Address: KOLISCH HARTWELL, P.C., 520 S.W. YAMHILL STREET, SUITE 200, PORTLAND, OR, 97204, US

(21) Appl. No.: 10/294,462

(22) Filed: Nov. 13, 2002

Related U.S. Application Data
(60) Provisional application No. 60/335,615, filed on Nov. 13, 2001.

(51) Int. Cl. A61B 5/00

(52) U.S. Cl. 600/300

(57) ABSTRACT

A system and method provide medical personnel with additional information about their patients. The system typically includes a portable computing device, communication link, printer, and/or fileserver. The method typically includes administering one or more healthcare screens from the portable computing device. On the portable device, the method typically includes displaying questions and appropriate answer choices for a patient to select from. In response to at least some of the questions, the method also typically includes displaying a next question and answers choices, the next question being useful in providing additional information about the patient. The method also typically includes scoring the healthcare screen according to standard scoring practices and printing or displaying a summary of the results. In addition, the method typically includes sending the summary to a printer and/or file server. The method may further include administering additional screens based on the score of the initial screen.

### CROSS-REFERENCE TO RELATED APPLICATIONS

This application claims priority to U.S. Provisional Patent Application Serial No. 60/335,615, filed Nov. 13, 2001 and entitled “A System and Method for Obtaining Patient Information”.

### TECHNICAL FIELD

The present invention relates generally to healthcare information and more particularly to a system, and method used for obtaining additional patient information during healthcare screens.

### BACKGROUND AND SUMMARY OF THE INVENTION

People who become sick or ill generally seek treatment at a hospital, urgent care facility, practitioner offices or clinics, or other similar medical setting. Treatment by a doctor, nurse, or other related medical personnel typically begins by performing an initial evaluation of the affected individual's medical condition. This evaluation typically includes the affected individual, or patient, responding to one or more healthcare screens, or sets of questions that address various issues related to the patient's condition. The screening process typically involves filling out one or more written questionnaire forms. Results of the screen(s) inform the medical personnel about the patient's symptoms, previous medical history, and any other matters related to the patient's condition. They are also typically used as an aid for diagnosis and treatment, however the screen itself is not interactive and cannot adjust to how a patient is responding to certain questions. This may result in the loss of valuable information relating to the patient or their condition, and may affect the ability to accurately diagnose and treat the patient. Therefore, it is desirable to provide a system and method that allows medical personnel to obtain additional information about their patients to improve diagnosis and treatment.

### SUMMARY OF THE INVENTION

A system and method are provided that are useful for providing medical personnel with additional information about their patients. The system typically includes a portable computing device, communication link, printer, and/or fileserver. The method typically includes administering one or more healthcare screens from the portable computing device. On the portable device, the method typically includes displaying questions and appropriate answer choices for a patient to select from. In response to at least some of the questions, the method also typically includes displaying a next question and answers choices, the next question being useful in providing additional information about the patient. The method also typically includes scoring the healthcare screen according to standard scoring practices and printing or displaying a summary of the results. In addition, the method typically includes sending the summary to a printer and/or file server. The method may further include administering additional screens based on the score of the initial screen.

### BRIEF DESCRIPTION OF THE DRAWINGS

FIG. 1 is a schematic view of a system according to one embodiment of the present invention.

FIG. 2 is a flowchart of a method for obtaining additional information about a patient according to one embodiment of the present invention.

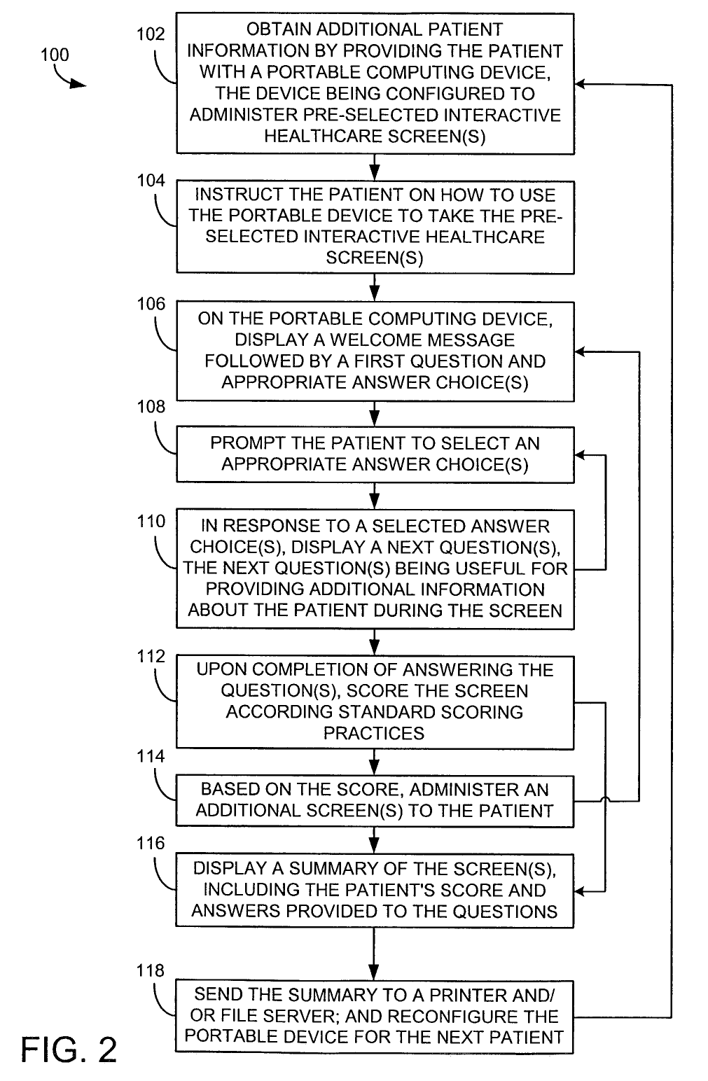

### DETAILED DESCRIPTION OF THE PREFERRED EMBODIMENTS

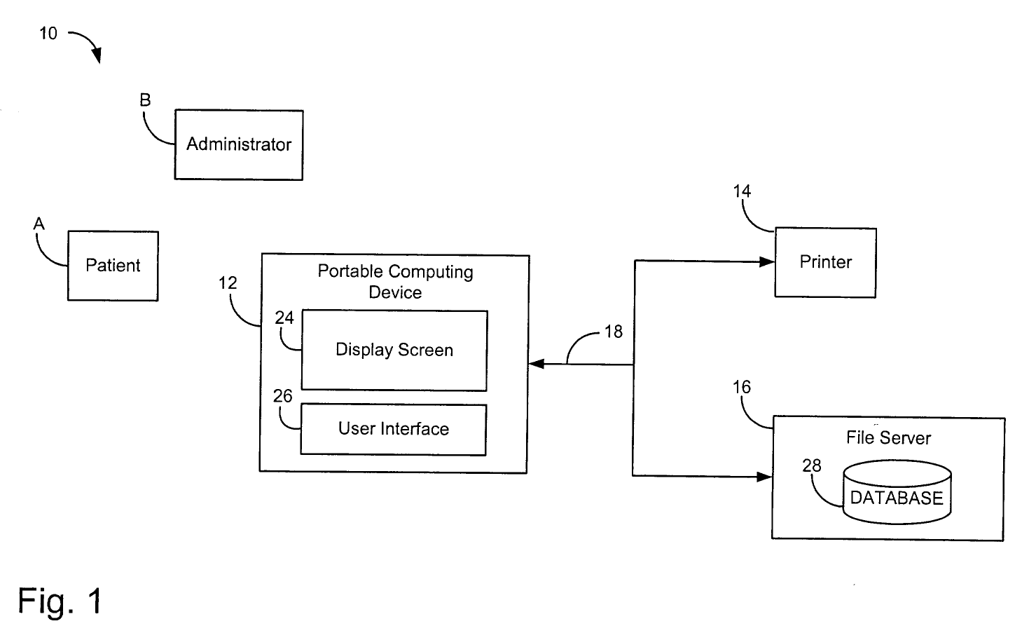

Referring initially to FIG. 1, one embodiment of a system to obtain additional patient information according to the present invention in generally shown at 10. System 10 includes portable computing device 12, printer 14, and file server 16 all connected by communication link 18. System 10 is configured so that administrator B can obtain additional information from patient A by administering one or more interactive healthcare screens. The interactive healthcare screen(s) typically include one or more questions and appropriate answer choices that are pre-selected by a healthcare provider or other relevant medical personnel.

Portable computing device 12 includes a display screen 24 and a user interface 26. Display screen 24 is typically a cathode ray tube (CRT) or flat panel display. User input device 22 is an interactive device that typically includes a keyboard and/or touch screen and/or mouse for the patient to use in response to the questions asked during the interactive healthcare screen. Device 12 typically includes healthcare screen software 26 configured to administer healthcare screens or other healthcare related questionnaires. Software 26 is updateable and may be further be configured to alter or modify healthcare screens or questionnaires based on general patient information such as gender, age, height, weight, or any other physical property that may not directly be related to a given patient's condition or illness. Software 26 also typically includes any applicable ranges and significant thresholds that may relevant to a patient's score or response to a specific question(s).

Printer 14 is typically configured to print documents sent from portable device 12. Printer 14 may be local, or remote, connected to device 12 via the internet, a dial-up connection, or other similar type of remote connection.

File server 16 typically includes database 26 that is configured to store the results of completed healthcare screens, healthcare screen summaries and/or any additional patient information sent from portable device 12. File server 16 may be local or remote, connected to device 12 via the internet, a dial-up connection, or other similar type of remote connection.

Referring next to FIG. 4, a method for obtaining additional patient information according to one embodiment of the present invention is generally shown at 100.

At 102, the method typically includes administrator B providing patient A with portable computing device 12, the device being configured to administer an interactive healthcare screen(s).

At 104, the method typically includes instructing patient A on how to use portable device 12 to answer questions presented on display screen 24.

At 106, the method typically includes portable device 12 displaying a welcome message to patient A followed by a first question and appropriate answer choices.

At 108, the method typically includes portable device 12 prompting patient A to select an appropriate answer choice for the question being asked.

At 110, the method typically includes portable device 12 receiving a response from patient A and displaying a next question, the next question being useful to obtain additional information about patient A.

At 112, the method typically includes portable device 12 scoring the administered healthcare screen upon its completion by patient A. Scoring is typically accomplished using standard scoring methods that are well known in the field.

At 114, the method may also include portable device 12 administering additional screens to patient A based upon the initial screen. Alternatively, portable device 12 may be initially configured to administer multiple healthcare screens, regardless of patient input.

At 116, the method typically includes portable device 12 displaying a summary of the healthcare screen(s). The summary may include patient A's score(s) and answers to health care screen questions. The summary may display the results of the screens individually or in combination to show answer trends for multiple screens. Alternatively, the summary may be displayed in any other configuration that provides the relevant medical personnel with additional patient information.

At 118, the method typically includes sending the summary from portable device 12 to printer 14 and/or file server 16 and reconfiguring portable device 12 for the next patient to be screened. Alternatively, the summary may be sent to other printers and/or file servers.

Although the invention has been disclosed in its preferred forms, the specific embodiments thereof as disclosed and illustrated herein are not to be considered in a limiting sense, because numerous variations are possible. The subject matter of the invention includes all novel and non-obvious combinations and subcombinations of the various elements, features, functions, and/or properties disclosed herein.

I claim:

1. A system for allowing a health care professional to obtain additional patient information about a patient, comprising:
a portable computing device;
effectively coupled to the device, at least one interactive healthcare screen subsystem usable by the professional based upon receiving information from the patient; and
a screen-scoring subsystem for scoring the interactive healthcare screen results.

2. A method for allowing a health care professional to obtain additional patient information about a patient, comprising:
choosing a patient;
performing a first interactive healthcare screen on the patient; and
scoring the first interactive healthcare screen.

3. The method of claim 2, further including the step of performing additional interactive healthcare screens based upon the results of the first interactive healthcare screen.
